(19) United States
(12) Patent Application Publication
(10) Pub. No.: US 2024/0002859 A1
(43) Pub. Date: Jan. 4, 2024

(54) PMO-BASED UTROPHIN::LET-7C MIRNA SITE BLOCKING OLIGONUCLEOTIDES (SBOS) FOR TREATING DUCHENNE MUSCULAR DYSTROPHY (DMD)

(71) Applicant: THE TRUSTEES OF THE UNIVERSITY OF PENNSYLVANIA, Philadelpnia, PA (US)

(72) Inventor: Tejvir S. S. KHURANA, Narberth, PA (US)

(73) Assignee: THE TRUSTEES OF THE UNIVERSITY OF PENNSYLVANIA, Philadelpnia, PA (US)

(21) Appl. No.: 18/253,919

(22) PCT Filed: Nov. 22, 2021
(86) PCT No.: PCT/US2021/060411

§ 371 Date: May 23, 2023

Related U.S. Application Data
(60) Provisional application No. 63/117,419, filed on Nov. 23, 2020.

(51) Int. Cl. C12N 15/113; A61P 21/00

(52) U.S. Cl. CPC C12N 15/1138; A61P 21/00; C12N 2310/141; C12N 2310/3233; C12N 2310/314

(57) ABSTRACT

The present disclosure provides methods and compositions for enhancing utrophin protein production by inhibiting binding of a Let-7c microRNA molecule to its binding site in the utrophin mRNA 3′-untranslated region (UTR). In particular, phosphorodiamidate morpholino oligonucleotide (PMO) site blocking oligonucleotides (SBOs) that inhibit Let-7c miRNA binding to its binding site in the utrophin mRNA 3′UTR. Moreover. methods of enhancing utrophin protein production in muscle cells can be used to treat muscular dystrophy and/or other myopathies.

### FIELD OF THE INVENTION

This disclosure relates to compositions and methods for treating myopathies, such as Duchenne Muscular Dystrophy (DMD). In one embodiment, compositions and methods are provided for enhancing utrophin protein expression in a subject by inhibition of microRNAs.

### BACKGROUND OF THE INVENTION

DMD is a lethal muscle wasting disease affecting approximately 1 in ˜5000 live-born males worldwide. DMD is caused by mutations in the DMD gene resulting in complete loss or extremely low expression of the dystrophin protein. Dystrophin (427 kDa) links the cytoskeleton with the extracellular matrix and is a key component of the dystrophin glycoprotein complex (DGC). During muscle contraction and relaxation cycles, dystrophin provides structural integrity to the myofiber. Loss of dystrophin and the concomitant destabilization of DGC, is thought to result in sarcolemmal fragility, altered membrane protein related signaling and defective muscle regeneration. Increased muscle damage leads to cycles of abortive muscle degeneration and regeneration accompanied by chronic inflammation. Eventually, contractile units are replaced with fibro-fatty tissues leading to a severe degree of muscle weakness and wasting. The disease progression typically leads to loss of ambulation in the carly teenage years and respiratory and cardiac failure by the fourth decade of life.

A promising therapeutic approach for DMD is upregulating utrophin which is an autosomal paralog of dystrophin. Utrophin could functionally compensate for the lack of dystrophin as it has a high degree of structural and functional similarity. The major utrophin isoform in myofibers, utrophin-A, is enriched at the neuromuscular and myotendinous junctions of adult muscles, and at the sarcolemma of regenerating myofibers. Utrophin upregulation has been obtained with small molecules such as heregulin, nabumetone, SMT C1100; artificial transcription factors and by regulating miRNAs that repress UTRN expression. miRNAs are short (˜22 nt) noncoding RNAs which, in association with RNA induced silencing complex (RISC), target complementary binding sites mostly within 3′UTRs of mRNAs. miRNAs are involved in post-transcriptional regulation of gene expression, typically by interfering with the stability and/or translation of target mRNAs. Previously, 2′-O-methyl-phosphorothioate (2OMePS) site blocking oligonucleotides (SBOs) have been used to block the Let-7c binding site in the UTRN 3′-untranslated region (UTR) to obtain upregulation of utrophin protein as well as functional improvement in mdx mice. However, the chemical moiety bas limitations as the charged nature of 2OMePS leads to suboptimal pharmacokinetic (PK) properties and therapeutic efficacy in terms of drug development. Clinical trials with dystrophin exon 51 skipping 2OMePS antisense oligonucleotides (AONs) have reported improvements in DMD; however, they were also accompanied by some side effects, e.g., proteinuria and moderate thrombocytopenia, that hampered progress.

Thus, a need exists for improvement in compositions for and methods of upregulating utrophin protein for the treatment of myopathies or utrophin-mediated diseases.

### SUMMARY OF THE INVENTION

In this disclosure, five Let-7c PMO-based SBOs (˜24-29bp) targeting different overlapping regions of a Let-7e binding site in the UTRN 3′UTR were designed in order to develop additional utrophin upregulation-based therapeutic strategies. These Let-7c PMO-based SBOs differ in sequence and chemical composition compared to previously described 2OMePS-based SBOs. A variety of biochemical, molecular, and morphological methods were used to rank order and test their ability to upregulate utrophin and ameliorate dystrophic phenotype in mdx mice. The results show that Let-7c PMO-based SBOs are a promising utrophin-mediated therapeutic approach for treating DMD (see FIG. 1).

In one aspect, the present disclosure relates to methods and compositions for enhancing utrophin protein production in a cell by inhibiting binding of utrophin microRNA.

In one aspect, provided herein are methods of treating a muscle disease or muscular dystrophy (e.g., Duchenne Muscular Dystrophy (DMD)) in a subject by administering to the subject an effective amount of an oligonucleotide that specifically hybridizes to a microRNA binding sequence in a utrophin mRNA 3′-untranslated region (UTR) and inhibits the binding of the microRNA to the utrophin mRNA 3′-UTR, wherein the microRNA can be Let-7c, miR-133b, miR-150, miR-196b, miR-206, miR-296-5p, or a combination thereof. The oligonucleotide can be designated as an antisense oligonucleotide or antisense molecule of the microRNA. Thus, in one embodiment, the oligonucleotide can be a Let-7c antisense oligonucleotide, an miR-133b antisense oligonucleotide, an miR-150 antisense oligonucleotide, an miR-196b antisense oligonucleotide, an miR-206 antisense oligonucleotide, an miR-296-5p antisense oligonucleotide, or a combination thereof.

In another aspect, provided herein are pharmaceutical compositions that include an oligonucleotide that specifically hybridizes to a microRNA binding sequence in a utrophin mRNA 3′-untranslated region (UTR) and inhibits the binding of the microRNA to the 3′-UTR of utrophin mRNA, wherein the microRNA can be Let-7c, miR-133b, miR-150, miR-196b, miR-206, miR-296-5p, or a combination thereof. In one embodiment, the oligonucleotide is present in an amount effective to block or interfere with the binding of the microRNA to the utrophin 3′-untranslated region (UTR). In one embodiment, the oligonucleotide can be a Let-7c antisense oligonucleotide, an miR-133b antisense oligonucleotide, an miR-150 antisense oligonucleotide, an miR-196b antisense oligonucleotide, an miR-206 antisense oligonucleotide, an miR-296-5p antisense oligonucleotide, or a combination thereof.

In one aspect, provided herein are methods of treating a muscle disease or muscular dystrophy (e.g., Duchenne Muscular Dystrophy (DMD)) in a human subject by administering to the subject an effective amount of an oligonucleotide that specifically hybridizes to a Let-7c microRNA binding sequence in a utrophin mRNA 3′-untranslated region (UTR) and inhibits the binding of the Let-7c microRNA to the utrophin mRNA 3′-UTR. In some embodiments, the oligonucleotide has a sequence that comprises a nucleic acid sequence set forth in SEQ ID NOs: 1-5. In one embodiment, the oligonucleotide has a sequence that comprises the nucleic acid sequence of SEQ ID NO: 5.

In another aspect, provided herein are pharmaceutical compositions that include at least one pharmaceutically acceptable excipient and an oligonucleotide that specifically hybridizes to a Let-7c microRNA binding sequence in a 3′-untranslated region (UTR) of a utrophin mRNA and inhibits binding of the Let-7c microRNA to the utrophin mRNA 3′-UTR, wherein the oligonucleotide is present in an amount effective in a human subject to inhibit binding of Let-7 microRNA to its utrophin mRNA 3′-UTR binding sequence. In some embodiments, the oligonucleotide has a sequence that comprises a nucleic acid sequence set forth in SEQ ID NOs: 1-5. In one embodiment, the oligonucleotide has a sequence that comprises the nucleic acid sequence of SEQ ID NO: 5.

In one aspect, provided herein are methods for treating a muscle disease or muscular dystrophy (e.g., Duchenne Muscular Dystrophy (DMD)) in a subject by administering to the subject an effective amount of a composition that inhibits binding of utrophin microRNA to the utrophin mRNA 3′-UTR. Also provided herein are the methods for reducing the symptoms associated with a muscular dystrophy (e.g., DMD) in a subject by administering to the subject an effective amount of a composition that inhibits binding of utrophin microRNA to the utrophin mRNA 3′-UTR.

In another aspect, provided herein are pharmaceutical compositions that include an effective amount of an agent that inhibits binding of utrophin microRNA to the utrophin mRNA 3′-UTR. For example, the agent can be a Let-7c antisense molecule, an miR-196b antisense molecule, an miR-133b antisense molecule, an miR-150 antisense molecule, an miR-206 antisense molecule, an miR-296-5p antisense molecule, or a combination thereof.

### BRIEF DESCRIPTION OF THE FIGURES

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings.

FIG. 1 is a schematic representation of the PMO-based Let-7c SBO strategy to alleviate Let-7c miRNA repression of the utrophin gene. The left panel shows Let-7c miRNA-mediated post-transcriptional repression of utrophin in control PMO treated mice. The right panel shows Let-7c PMO-mediated blocking of miRNA binding to UTRN 3′UTR, resulting in higher utrophin expression and improvement in dystrophic pathophysiology.

FIGS. 2A-2D show that Let-7c PMO SBO upregulates utrophin expression in the C2C12 cell line. FIG. 2A is a schematic diagram showing the relative position of the Let-7c binding site of five different Let-7c PMO SBOs in UTRN 3′UTR. FIG. 2B shows luciferase expression in C2C12UTRN5′luc3′ reporter cell line after treatment with 0.1 μM, 0.5 μM, 1 μM and 3 μM of control or five different Let-7c PMO SBOs for 24 hrs. Data shown as percentage of normalized luciferase expression compared with control PMO-treated cells (n=3). FIG. 2C shows endogenous utrophin expression in C2C12 cell line after treatment with 0.5 μM, 1 μM, 3 μM and 5μM of control or five different Let-7c PMO SBOs for 48 hrs. FIG. 2D shows quantification of endogenous utrophin expression in C2C12 cell line as percentage of normalized utrophin expression compared with control PMO treated cells (n=3). Each bar represents mean±SEM. Statistical analysis performed by Two-way ANOVA Tukey's multiple comparisons test. *p<0.1, **p<0.01, ***p<0.001, ****p<0.0001.

FIGS. 3A-3C show higher expression of utrophin in mdx mice after five weeks of treatment with 80 mg/kg/wk S56 PMO. FIG. 3A is a diagram of the experimental pipeline adopted for the study. FIG. 3B are western blots showing higher level of utrophin expression in gastrocnemius and soleus muscles of S56 PMO-treated mdx mice. Samples from three different max mice treated with control or S56 PMO were shown. FIG. 3C shows quantification of normalized utrophin level in gastrocnemius and soleus muscle in control and S56 PMO-treated mdx mice (n=6 mice for both groups). Data shown as percentage of normalized utrophin level compared to control PMO-treated mdx mice. Each bar represents mean±SEM. Statistical analysis performed by Mann-Whitney nonparametric test, **p<0.01.

FIGS. 4A-4B show immunohistochemistry of mdx TA muscles treated with S56 PMO have higher sarcolemmal expression of utrophin. FIG. 4A: TA muscles were stained with a-bungarotoxin (green), utrophin antibody (red) and wheat germ agglutinin (cyan). The control PMO and S56 PMO-treated cryosections were also stained with utrophin pre-immune sera as control. Scale bar=100 μm. The figures showed synaptic expression of utrophin in control PMO, and both synaptic and extra-synaptic sarcolemma-associated expression of utrophin in S56 PMO-treated mice. FIG. 4B shows quantification of utrophin expression in sarcolemma normalized with WGA expression. S56 PMO-treated muscles showed significantly higher expression of utrophin (****p=0.0001, n=4 mice for both groups). Statistical analysis performed by Mann-Whitney nonparametric test.

FIGS. 5A-5F show H&E staining of cryosections from control PMO or S56 PMO-treated mdx mice. FIG. 5A shows representative H&E staining image of whole EDL cryosections (scale bar=100 μm). FIG. 5B shows regions highlighted by the white boxes in FIG. 5A were magnified (3×) (scale bar=25 μm). FIG. 5C shows representative H&E staining of diaphragm cryosections (scale bar=200 μm). Control PMO-treated EDL and diaphragm muscle cryosections show regenerated myofibers (arrow) and immune cell infiltration (arrowhead) and S56 PMO treatment appeared to alleviate symptoms. FIGS. 5D-5E show quantification of centrally nucleated EDL and diaphragm muscle fibers of mdx mice treated with control PMO or S56 PMO. The graphs show significant decrease in the percentage of CNFs of EDL and diaphragm muscles in S56 PMO-treated mdx mice (**p=0.002, n=10 mice for both groups of EDL muscles, *p=0.0140, n=6 mice for both groups of diaphragm muscles). FIG. 5F shows serum CK levels in S56 PMO-treated mice were significantly lower than control PMO-treated mdx mice (*p=0.02, n=10 mice for both groups). Each bar represents mean±SEM. Statistical analysis performed by Mann-Whitney nonparametric test.

FIGS. 6A-6I show utrophin protein and RNA expression in mdx mice treated with 80 mg/kg/week control PMO or S56 PMO for five weeks. Representative western blots show higher utrophin protein expression in S56 PMO treated TA (FIG. 6A), EDL (FIG. 6B) and diaphragm (FIG. 6C) tissues. Quantification of the normalized utrophin protein expression in control PMO and S56 PMO-treated TA (FIG. 6D), EDL (FIG. 6E) and diaphragm (FIG. 6F) tissues. Data shown as percentage of normalized utrophin protein expression compared with control PMO-treated mice. Each bar represents mean±SEM (n=6). Statistical analysis performed by Mann-Whitney nonparametric test with statistical significance level set at α≤≤0.05. (*p=0.04 for TA, *p=0.03 for EDL and **p=0.003 for diaphragm). Utrophin RNA expression in the corresponding TA (FIG. 6G), EDL (FIG. 6H) and diaphragm (FIG. 6I) tissues. Data shown as fold change in normalized utrophin RNA expression compared with control PMO-treated mdx mice tissues. Each bar represents mean±SEM (n=4). Statistical analysis performed by Mann-Whitney nonparametric test with statistical significance level set at α≤0.05. (p=0.22 for TA, *p=0.02 for EDL and p=0.2 for diaphragm).

FIGS. 7A-7B show utrophin RNA expression in the gastrocnemius and soleus muscles of mdx mice treated with 80 mg/kg/week control PMO or S56 PMO for five weeks.

Data shown as fold changes in normalized utrophin RNA expression in S56 PMO-treated mdx mice compared with control PMO-treated mdx mice. Each bar represents mean±SEM (n=4). Statistical analysis performed by Mann-Whitney nonparametric test with statistical significance level set at α≤0.05. (p=0.11 for gastrocnemius, *p=0.02 for soleus).

FIGS. 8A-8B show restoration of β-DG in S56 PMO-treated mdx mice. Representative western blots show expression of β-DG in the wild type mice and absence in mdx mice treated with control PMO. S56 PMO-treated mdx mice show partial restoration of β-DG expression compared with control PMO-treated mdx mice in TA and EDL tissues.

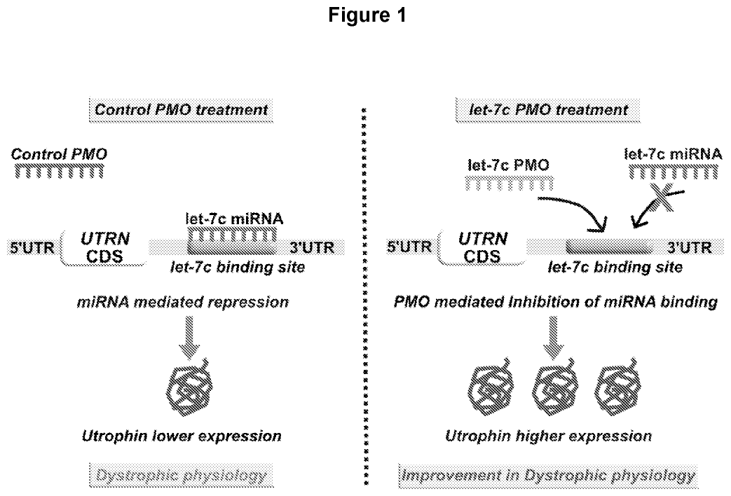

### DETAILED DESCRIPTION OF THE INVENTION

Upregulation of utrophin, a dystrophin related protein, is considered a promising therapeutic approach for Duchenne Muscular Dystrophy (DMD). Utrophin expression is repressed at the post-transcriptional level by a set of miRNAs, among which Let-7c is evolutionarily highly conserved. In this disclosure, PMO-based SBOs complementary to the Let-7c binding site in UTRN 3′UTR were designed to inhibit Let-7c interaction with UTRN mRNA and thus upregulating utrophin. The C2C12UTRN5′luc3′ reporter cell line in which the 5′- and 3′-UTRs of human UTRN sequences flank luciferase was used for reporter assays. The C2C12 cell line was used for utrophin western blots to independently evaluate the site blocking efficiency of the series of Let-7c PMOs in vitro. Treatment of one-month old mdx mice with the most effective Let-7c PMO (i.e. S56) resulted in ca. two-fold higher utrophin protein expression in skeletal muscles and improvement in dystrophic pathophysiology in mdx mice in vivo. In summary, results presented herein show that PMO-based Let-7c SBO has applicability for upregulating utrophin expression as a therapeutic approach for DMD.

In one aspect, provided herein is a method of enhancing utrophin protein production in a cell, comprising the step of inhibiting binding of utrophin microRNA to the utrophin mRNA 3′-UTR. In one embodiment, the cell is a muscle cell.

In another aspect, provided herein are methods of treating a muscle disease or muscular dystrophy (e.g. Duchenne Muscular Dystrophy (DMD)) in a human subject by administering to the subject an effective amount of an antisense oligonucleotide that specifically hybridizes to a Let-7c microRNA binding sequence in a utrophin mRNA 3′-untranslated region (UTR) and inhibits binding of Let-7c microRNA to the utrophin mRNA 3′-UTR. In some embodiments, the oligonucleotide comprises a nucleic acid sequence set forth in SEQ ID NOs: 1-5. In one embodiment, the oligonucleotide comprises the nucleic acid sequence of SEQ ID NO: 5.

In another aspect, provided herein are pharmaceutical compositions that include an antisense oligonucleotide that specifically hybridizes to a Let-7c microRNA binding sequence in the utrophin mRNA 3′-untranslated region (UTR), wherein the antisense oligonucleotide is present in an amount effective in a human subject to inhibit the binding of Let-7 microRNA to its utrophin mRNA 3′-UTR binding sequence. In some embodiments, the oligonucleotide comprises a nucleic acid sequence set forth in SEQ ID NOs: 1-5. In one embodiment, the oligonucleotide comprises the nucleic acid sequence of SEQ ID NO: 5.

In one embodiment, provided herein is a method of enhancing utrophin protein production in a cell, comprising contacting a microRNA molecule that binds a utrophin mRNA with an inhibitor.

In one embodiment, provided herein is a method of enhancing utrophin protein production in a cell, comprising contacting a microRNA molecule that binds a utrophin mRNA 3′-UTR with an antisense molecule which inhibits binding of the microRNA to a utrophin mRNA 3′-UTR, thereby enhancing utrophin protein production in a cell, e.g., a muscle cell. In one embodiment, provided herein is a method of enhancing utrophin protein

production in a cell, comprising contacting a muscle cell specific microRNA molecule that binds a utrophin mRNA 3′-UTR with an inhibitor, thereby enhancing utrophin protein production in a cell. In one embodiment, the cell is a muscle cell.

In one embodiment, provided herein is a method of enhancing utrophin protein production in a cell, comprising the step of contacting a muscle cell specific microRNA molecule that binds a utrophin mRNA 3′-UTR with an antisense molecule which inhibits the binding of the microRNA to a 3′-UTR utrophin mRNA, thereby enhancing utrophin protein production in a cell. In one embodiment, the cell is a muscle cell.

The wild-type mouse (Mus musculus) utrophin mRNA sequence can be found in GenBank with GenBank Accession #AK035043.1; its 3′-UTR has the following sequence:

(SEQ ID NO: 7)
TGAGCATCTATCCAGCCAGCCAACATTTCCCGACCTTCAGTATTGCCCTC
TTCTGCAAATGCCAATCCCAAGACCCATTCAACCCCAAAGCTCCGTGGCT
CCACGACACAAGCTGTTGAGTGCTTACTGGGTGTTCTACTGAGGGAACCA
AACACTGACTATCCAAAGAGAAAAGGATATTTTGGTTTTCTAATAACGTA
TATTATTGTTTTCTTCTCCCCTTTCTATGCAACTGTAAATTAATGAACAG
AGAAGTATTTGGAGGTGGTAAAGCATTTGTCACTGATTTGTATAATATAT
ACAGCCATGGGAAAGTGGGTGGGGGCTTTCTAATATGAAACTGTCTTTTT
AATAACCAAGAGAAAAAATTGCATAAGAATTAGACCACTTTACATTATTA
CATTCCTTCTGCTGTTCACATTAACCTTGTACAATAACTTCACTTATTAT
TTGACTGTTTTACCATTATGTTTTGGTTATTTATAAATTTATCAGCCATA
CAAACAAATAGATTCTATGTATTTGTTTCTATAATCTGGCCAAATTCCTA
AGTTCATATATTTGAATCAAATATTTTACATATGTGGAGTAGGCAGGCAT
TCTGAAGATACTATTTAACTTTAGTTGACGTCACACACACCATCCTTTAG
TAACCACTGGATGACTACACTAAAAATCCTGTGGACTTTAACGGCAAGCT
GCTGGGGTATTTTTCCTCCTGTTTTTATTCCTTTTTTGTAAGTAGATCTT
GACGTCTTTATTTATTTCATCTTGCAATCTCTATAATAAAGAAGACTGTA
TTGTAATAGTCTCAAAAAATTATTTTACCAAGGGTTACCATTTAAGCATA
TTTTCATTTTGATTCAGAAACCAAAGTTGGTACAACCTCTCCTAGTACAT
GCAACCTTGGTTTTCATGAGAAAACACACGGCAGGCCTTTGCCCATTGTG
AGGAGAGCACACATCATGCTCTTCAGTTTCCTTTGAATAGACTTTTATTG
TTGTTTTTGTATTTTTCGAGTCCTGTGTAAGTTTTGAAAGCTCTGGTTGT
TTCCTTTGTGAAAGCAGGCAGATACTTAGTTGGCTGTCTCATTTGAAGCT
TTGGAGCAGATAGTCAGATGTCTCATGACCCCTCACTTGGCCAGCAGCAC
ATCCGAGAAGGATGTCACTCACAAGCCTACACCACGGCTTCTCTAGAATG
AAATCAGTGCTCGGATGATTGTATCCCTGCCTCTACTTCTGAGTGTGTTC
AACTAGGTATTGGCTTCTTTTTCTTTTTCTTTTCTTTTTTTTTTAATTTA
ACACTTAATTGCCGATTTTAGAGAAACCAAAAATAAAGGTGAAGGTAATA
TGTTTTGATTCAAACATATATGCTTTTAAACATCAGACATGCTAACTTTG
GTTCTCTTTACTGGAATCTGGCCCAGAGGAGGTGAAATTTAGAAATGTTA
TTCTTTAGATGGGTGGGTGGGTTGGGGGGCCAAGGGTGTCTATTTTCCAG
CATTAGATATTTTTGAGACGAAGAAAATTGTTTTATATAAGGGGAGAGCC
ATGATCACCTTTCTACCTCAGAACCACCTTCCTCCATTGTGTTGGACATA
GCTTTATATGCCGCAGTGTGCAAAACCTAGGGCTGTAGTCAGGCCTTTCC
ATACCCAGGAAGCACCTGTGTAAAGAAGATCAACAGAAACTCCCGGAACT
CAGAACCCCAAGTTGTAGATTTGGTGTCGTCCTTGTTCTTGCTTTGAGGA
GTCATGTATTCTTTTATTTCCTGCCTGTATTTGTATGCAAAATGATCTCT
ATCTGCTATTACAGAAAAAGCTACACAAAACACTACATTGTAACCTTCTG
AGTAATAAATAAGAGGAAATATATTACAGTAACCATGATGAGAAATAAGT
GTATTGTTCTTTTGAAATATGTGGTTAATCGCAGACTGTCATCTAATCTG
TTACATACCGTATTTTTCATCCTGAATAAAAGTAATTTTAACACAAAATG
ACTTTGATGTTTGGCTGTGTTCAGCTGATGAAATCAGATCTCTGAATGTA
TGTGATGAAAGCTAACTATAAGATGATCTATATTCTGATAAATCTAAATA
TTTTCTGAAACTCTCTCTTATACATTAATCTAGTCTCCATTCACTCATTA
TCTCTCTCTCCTTTCTTGCATATAAATATGATTATATATTTTTCAATTTC
CTGTACAAATCAGAGTCTTATTACTAGGGAAAATGGATGTTATAAGTACA
TTCCTAAAGCCCATTGGGCCTTCATTTTTATAACTTGGAGCTACTGAGAT
TTATCAGGTTACTCTCTCAAATCCACTTTCATCACTAGACTCATAGTTTT
CTATGTATCTATATTATTATAACTAAATAAAAATATACATG.

The wild-type human (Homo sapiens) utrophin mRNA sequence can be found in GenBank with GenBank Accession #NM 007124.2; its 3′-UTR has the following sequence:

(SEQ ID NO: 8)
TGAAGTATTCATCCGGCCAACCAATGTTTCCTGACGTACAGTGTTGCCCT
TTTCAGCAAATGCCAATTCCAAGTTCCATTAAATCAGAAGCTCCATGGCT
CCTTGGCCCACGATGTTGAGTGCTGACTGTGTGTTCTACTGAAAGAGTAA
AACACTGACTATCCAAAGAGAAATGGATATTTTGTTTTTATAATAACCAT
ATATTATTGTTTTCTTCTTCCCTTTCTATGCAAGTGTAAATTAATGAACA
GAGAGGTATTTGGAAATGGTAATACATTTGTCACGGATTTGTATAATGTA
TACAGCATTGGGAAAGTGGGTGGGGGCTTTCTAATATGATACCGTCTTTT
TAATAACTATGACAAAGCTTACATAAGAATTAGAAGACCACTTTACATTT
TTACATTCCTTCTGCTGTTCATATTAACCTTGCACAATTACTTCATTTTT
TCTTTGACTCTTTTACCACAATGTTTTGGTTATTTATAATTTATCAGCCA
TATGTTTATCAGCCATATAACCAACTAGATCCCAAATAGATCCATGTATT
TGTTTCCGTGATTTGGCCACATTAATAAATTCATAAATTTCAATCAAATA
TCTTATATATACACACATATGGTTTAAGCTACAGCCCTGTGTATGCCGTT
TAACTTTATTTGACGTTGCCCACTTACTTCTTTGCTGACCACTTGGATAA
CCGTAATAAAAATCCTATAAGCCTAAATGGCATTTCTTTTGGGATATTTT
TCCTGCATTTTATTCCCTTTTTATATAAGTAGGAATTAATTATTTATTTT
ATGTCTTAATCTATTTGATAAAGAAGACTACATTATAATAATCTCAAAGA
TCATATTACCAAAGGTTGCCCACTTGAGCATATTTTCATTTTGACACAGA
AACAAAATTTAGTACAACCTTTCCTAGTTCCCATGTCTTGATTTTCATCA
TTACATGCACAGCAGACCTTTACCTATTGTGATACCAGAACACATCATTG
TCTTTGGTTCCCTTCAAAGAGAATTTTATTGTTGTTTTGTATTTTCAAGT
CCTTAATAGTTCTTGAAACTCCTAGTTGTTTTCTTGTTGAAAGCAGACAC
ACATTTAGTGCACGGCTTATTTTACCTTTCGGGTGAAAGATCAGATGTTT
TTATACCCTTCACTTGATCAATATATTTGGAAAGAATGTTTATCAAAAGT
CTATGTCACTGCTTCTACAGAAGAATGAAATTAATGCTTAGGTGATGGTA
CCTCCACCTACATCTTTTTGAGTGCATTCAATTATGTATTTTGGTTTAGC
TTCTGATTTAACATTTAATTGATTCAGTTTAAACATGTTACTTAATTAGC
AAATGTAGAGGAACCAAAAAAAGGTGAAAATAATATGTTTTGATTCAAAC
CTAAAGACATAAAAACATAAAGACATTTTAACTTTGGGTTCTCTTTAGCT
GGGATCTGGCCAGAAGGAGGCTTAAAGTTAGAAATTGCTATTATTTTAGA
ATAGGTTGGGTGGGTTGGGGGGCAAGGGTGTCTATTTGCAGCAGAGATAT
TTTGAAAAGAAGAAAATTGTTTTATATAAAAAGGAAAGCCATGACCACCT
TTCTACCTCAGATCCATCTTCATCCATTGCATTGGAAACTGCTTTATGCT
GCTGCAGTCTGCAAAGTCTAGAGCTTTTATCAGGCCATGTCATACCCAAG
AAAGCACCTATTTAAAGAAAAAACAATTCCCTGAGCTCTCAACTCCAAGT
TGTAGATTTGGTGTCTTCCTTGTTCTTACTTTAAAAAGTCATGTGTTAAT
TTTTTTTCTGCCTGTATTTGTATGCAAAATGTCCTCTATCTGCTATTAAA
GAAAAGCTACGTAAAACACTACATTGTAACCTTCTAAGTAATAATAAATA
AAAAGAAATATATTGCAGTAACAATGGGAAGTAAGTATGTAGTTCTTTTG
AAATATGTGGTAAAGAACTAATCACAGACTATCATCTAATCTGGTTACAT
ATTGTATTTTTCATCCTGAATAAAAGTAATTTTAACACAAAAAAA.

In one embodiment, the utrophin mRNA 3′-UTR nucleic acid sequence is a homolog, a variant or a functional fragment of SEQ ID NO: 7. In one embodiment, the utrophin mRNA 3′-UTR nucleic acid sequence is a homolog, a variant or a functional fragment of SEQ ID NO: 8.

Without wishing to be bound by theory, a microRNA of the invention inhibits utrophin protein production by binding to a utrophin 3′-UTR binding sequence.

In mouse mRNA, the Let-7c binding sequence in utrophin 3′-UTR is: 5′-AGCCATGATCACCTTTCTACCTCA-3′ (SEQ ID NO: 9).

In human mRNA, the Let-7c binding sequence in utrophin 3′-UTR is: 5′-AGCCATGACCACCTTTCTACCTCA-3′ (SEQ ID NO: 10).

In one embodiment, the cell is a skeletal muscle cell. In one embodiment, the cell is a smooth muscle cell. In one embodiment, the cell is a satellite muscle cell. In one embodiment, the cell is a cardiac muscle cell.

In one embodiment, the microRNA molecule is a muscle cell specific microRNA molecule. In one embodiment, the microRNA molecule binds to utrophin. In one embodiment, the microRNA molecule is complementary to a utrophin RNA sequence. In one embodiment, the microRNA molecule is complementary to a 3′-UTR of a utrophin RNA sequence. In one embodiment, the microRNA molecule decreases the levels of utrophin protein. In one embodiment, the microRNA molecule decreases utrophin protein levels without decreasing utrophin mRNA levels. In one embodiment, the microRNA molecule targets utrophin-A IRES. In one embodiment, the microRNA molecule targets utrophin-A IRES in a muscle cell. In one embodiment, the microRNA molecule represses utrophin-A IRES activity.

In one embodiment, the microRNA molecule is miR-206. In one embodiment, the microRNA molecule is Let-7c. In one embodiment, the microRNA molecule is miR-196b. In one embodiment, the microRNA molecule is miR-133b. In one embodiment, the microRNA molecule is miR-150. In one embodiment, the microRNA molecule is miR-296-5p.

The sequence of Let-7c microRNA (mouse and human) is: 5′-UGAGGUAGUAGGUUGUAUGGUU-3′ (SEQ ID NO: 11).

In one embodiment, the microRNA molecule Let-7c comprises the sequence of

miRBase accession number MI0000064. In one embodiment, the microRNA molecule Let-7c comprises the sequence of miRBase accession number MI0000559. In one embodiment, the microRNA molecule Let-7c comprises the sequence of miRBase accession number MI0000560. In one embodiment, the microRNA molecule Let-7c comprises the sequence of miRBase accession number MI0000830. In one embodiment, the microRNA molecule Let-7c comprises the sequence of miRBase accession number MI0000831. In one embodiment, the microRNA molecule Let-7c comprises the sequence of miRBase accession number MI0001174. In one embodiment, the microRNA molecule Let-7c comprises the sequence of miRBase accession number MI0001866. In one embodiment, the microRNA molecule Let-7c comprises the sequence of miRBase accession number MI0001867. In one embodiment, the microRNA molecule Let-7c comprises the sequence of miRBase accession number MI0002445. In one embodiment, the microRNA molecule Let-7c comprises the sequence of miRBase accession number MI0004886. In one embodiment, the microRNA molecule Let-7c comprises the sequence of miRBase accession number MI0005124. In one embodiment, the microRNA molecule Let-7c comprises the sequence of miRBase accession number MI0005454. In one embodiment, the microRNA molecule Let-7c comprises the sequence of miRBase accession number MI0007138. In one embodiment, the microRNA molecule Let-7c comprises the sequence of miRBase accession number MI0007152. In one embodiment, the microRNA molecule Let-7c comprises the sequence of miRBase accession number MI0007183. In one embodiment, the microRNA molecule Let-7c comprises the sequence of miRBase accession number MI0007184. In one embodiment, the microRNA molecule Let-7c comprises the sequence of miRBase accession number MI0007574. In one embodiment, the microRNA molecule Let-7c comprises the sequence of miRBase accession number MI0008076.

Utrophin upregulation is a therapeutic strategy for DMD. Normally, Utrophin-A expression is repressed through the 5′- and 3′-UTRs by >98% at the translational level. The Utrophin 5′- and 3′-UTR contains microRNA target sites. In one embodiment, the Utrophin 3′-UTR exhibits its inhibitory effect both on IRES and on cap-dependent translation. Inhibition of microRNAs that target Utrophin UTRs by blocking the microRNA binding site in the mRNA or by binding to the microRNA itself are therapeutic strategies for DMD.

In one embodiment, inhibiting a microRNA molecule comprises contacting a microRNA with a complementary antisense oligonucleotide sequence. In one embodiment, inhibiting a utrophin microRNA comprises contacting a utrophin microRNA molecule with a utrophin microRNA antisense molecule. In one embodiment, inhibiting a utrophin microRNA molecule comprises contacting a utrophin microRNA molecule with an antisense molecule that specifically binds to or hybridizes with the utrophin microRNA. An oligonucleotide “specifically hybridizes” to a target polynucleotide if it hybridizes to the target under physiological conditions, with a Tₘ substantially greater than 45° C., preferably at least 50° C., and typically 60° C.-80° C. or higher. Such hybridization preferably corresponds to stringent hybridization conditions. At a given ionic strength and pH, the Tₘ is the temperature at which 50% of a target sequence hybridizes to a complementary polynucleotide. Again, such hybridization may occur with “near” or “substantial” complementary of the antisense oligomer to the target sequence, as well as with exact complementarity.

In one embodiment, inhibiting Let-7c, miR-196b, miR-133b, miR-150, miR-296-5p, miR-206 or any combination thereof leads to utrophin upregulation. In one embodiment, an inhibitor of Let-7c, miR-196b, miR-133b, miR-150, miR-296-5p, miR-296 or any combination thereof is used as a Duchenne muscular dystrophy therapeutic agent.

In one embodiment, an antisense specific molecule comprises at least 3 consecutive nucleotides which are complementary to a utrophin microRNA molecule or a fragment thereof. In one embodiment, an antisense specific molecule comprises at least 3 consecutive nucleotides which are complementary to a muscle cell utrophin microRNA molecule or a fragment thereof. In one embodiment, an antisense specific molecule comprises at least 3 consecutive nucleotides which are complementary to a utrophin microRNA molecule or a fragment thereof as described herein. In one embodiment, an antisense specific molecule comprises at least 3 consecutive nucleotides which are complementary to any microRNA molecule represented by a miRBase accession number as described hereinabove or a fragment thereof. In one embodiment, an antisense specific molecule comprises at least 4 consecutive nucleotides which are complementary to any microRNA molecule represented by a miRBase accession number as described hereinabove or a fragment thereof. In one embodiment, an antisense specific molecule comprises at least 5 consecutive nucleotides which are complementary to any microRNA molecule represented by a miRBase accession number as described hereinabove or a fragment thereof. In one embodiment, an antisense specific molecule comprises at least 7 consecutive nucleotides which are complementary to any microRNA molecule represented by a miRBase accession number as described hereinabove or a fragment thereof. In one embodiment, an antisense specific molecule comprises at least 9 consecutive nucleotides which are complementary to any microRNA molecule represented by a miRBase accession number as described hereinabove or a fragment thereof. In one embodiment, an antisense specific molecule comprises at least 11 consecutive nucleotides which are complementary to any microRNA molecule represented by a miRBase accession number as described hereinabove or a fragment thereof.

In one embodiment, an antisense specific molecule comprises at least 3 consecutive nucleotides derived from the 5′-UTR or the 3′-UTR of a utrophin RNA molecule. In one embodiment, an antisense specific molecule comprises at least 4 consecutive nucleotides derived from the 5′-UTR or the 3′-UTR of a utrophin RNA molecule. In one embodiment, an antisense specific molecule comprises at least 5 consecutive nucleotides derived from the 5′-UTR or the 3′-UTR of a utrophin RNA molecule. In one embodiment, an antisense specific molecule comprises at least 6 consecutive nucleotides derived from the 5′-UTR or the 3′-UTR of a utrophin RNA molecule. In one embodiment, an antisense specific molecule comprises at least 8 consecutive nucleotides derived from the 5′-UTR or the 3′-UTR of a utrophin RNA molecule. In one embodiment, an antisense specific molecule comprises at least 10 consecutive nucleotides derived from the 5′-UTR or the 3′-UTR of a utrophin RNA molecule. In one embodiment, an antisense specific molecule comprises at least 12 consecutive nucleotides derived from the 5′-UTR or the 3′-UTR of a utrophin RNA molecule. In one embodiment, an antisense specific molecule comprises at least 14 consecutive nucleotides derived from the 5′-UTR or the 3′-UTR of a utrophin RNA molecule. For example, an antisense oligonucleotide derived from the 5′-UTR or the 3′-UTR of a utrophin mRNA encompasses sequences that are complementary to sequences in the 5′-UTR or 3′-UTR. In one embodiment, an antisense specific molecule comprises a complementary sequence to any microRNA molecule represented by a miRBase accession number as described hereinabove or a fragment thereof. In one embodiment, an antisense specific molecule comprises a homologous complementary sequence to any microRNA molecule represented by a miR Base accession number as described hereinabove or a fragment thereof.

A homologous complementary sequence is at least 60% homologous, at least 70% homologous, at least 80% homologous, at least 90% homologous, at least 95% homologous, at least 98% homologous, at least 99% homologous, or even 100% homologous.

In one embodiment, an antisense specific molecule comprises at least 3, at least 5, at least 7, at least 9, at least 11, or at least 13 consecutive nucleotides complementary to Let-7c.

In one embodiment, an antisense specific molecule comprises at least 3, at least 5, at least 7, at least 9, at least 11, or at least 13 consecutive nucleotides complementary to miR-196b. In one embodiment, an antisense specific molecule comprises at least 3, at least 5, at least 7, at least 9, at least 11, or at least 13 consecutive nucleotides complementary to miR-133b. In one embodiment, an antisense specific molecule comprises at least 3, at least 5, at least 7, at least 9, at least 11, or at least 13 consecutive nucleotides complementary to miR-150. In one embodiment, an antisense specific molecule comprises at least 3, at least 5, at least 7, at least 9, at least 11, or at least 13 consecutive nucleotides complementary to miR-296-5p. In one embodiment, an antisense specific molecule comprises at least 3, at least 5, at least 7, at least 9, at least 11, or at least 13 consecutive nucleotides complementary to miR-206.

In one embodiment, an antisense molecule is a synthetic peptide nucleic acid (PNA). In one embodiment, an antisense molecule is a LNA.

In another aspect, provided herein are antisense oligonucleotides effective to inhibit binding of Let-7 microRNA to its corresponding 3′-untranslated region (UTR) in utrophin mRNA. In one embodiment, the antisense oligonucleotide includes a nucleic acid sequence set forth in SEQ ID NO: 1, a fragment thereof, or a variant thereof. In one embodiment, the antisense oligonucleotide includes a nucleic acid sequence set forth in SEQ ID NO: 2, a fragment thereof, or a variant thereof. In one embodiment, the antisense oligonucleotide includes a nucleic acid sequence set forth in SEQ ID NO: 3, a fragment thereof, or a variant thereof. In one embodiment, the antisense oligonucleotide includes a nucleic acid sequence set forth in SEQ ID NO: 4, a fragment thereof, or a variant thereof. In one embodiment, the antisense oligonucleotide includes a nucleic acid sequence set forth in SEQ ID NO: 5, a fragment thereof, or a variant thereof. In some embodiments, a variant antisense oligonucleotide includes oligonucleotides where one or more additional bases have been added to and/or deleted from the 3′ and/or S′ end.

In another aspect, provided herein are compositions that comprise an antisense oligonucleotide described herein, wherein the oligonucleotide molecule is present in an amount effective to inhibit binding of Let-7 microRNA to its corresponding 3′-untranslated region (UTR) in utrophin. In another aspect, provided herein are compositions that comprise an antisense oligonucleotide described herein with at least one suitable excipient, for example, a pharmaceutically acceptable excipient, or an additive, known in the art.

In some embodiments, the antisense oligonucleotide is a synthetic oligonucleotide.

The antisense oligonucleotide can be synthesized by any suitable method known in the art. In some embodiments, the antisense oligonucleotide is a morpholino or phosphorodiamidate morpholino oligonucleotide (PMO) or Vivo-morpholino molecule.

Morpholinos and PMOs are known in the art and are synthetic molecules that are the product of a redesign of natural nucleic acid structure. See, e.g., Summerton et al., “Morpholino Antisense Oligomers: Design, Preparation and Properties”. Antisense & Nucleic Acid Drug Development 7 (3): 187-95. PMOs bind to complementary sequences by standard nucleic acid base-pairing. The structural difference between morpholinos and DNA is that, while morpholinos have standard nucleic acid bases, those bases are bound to morpholine rings instead of deoxyribose rings. In addition, PMOs are linked through phosphorodiamidate groups instead of phosphates. Replacement of anionic phosphates with uncharged phosphorodiamidate groups eliminates ionization in the physiological pH range, and thus morpholinos in organisms or cells are uncharged molecules. Vivo-Morpholinos are comprised of a Morpholino oligo with a unique covalently linked delivery moiety that is comprised of an octa-guanidine dendrimer.

Without wishing to be bound by theory, when an antisense oligonucleotide blocks binding of Let-7 microRNA to its corresponding binding sequence in the 3′-UTR of a utrophin mRNA, it is capable of enhancing utrophin production, and thereby treating Duchenne Muscular Dystrophy (DMD).

Antisense oligonucleotides may be made by any suitable method known in the art. For example, an antisense oligonucleotide is produced by a chemical process, for example by the chemical phosphoamidite method comprising sulfuration with tetraethylthiuram disulfide in acetonitrile (Tetrahedron Lett., 1991, 32, 3005-3008).

In one embodiment, provided herein is a method for treating or reducing the signs and symptoms associated with muscular dystrophy in a subject, the method comprising administering to the subject a composition that inhibits binding of a utrophin microRNA to the utrophin mRNA 3′-UTR.

Muscular dystrophy may refer to any type of muscular dystrophy. In one embodiment, the muscular dystrophy is Duchenne Muscular Dystrophy (DMD). In one embodiment, the muscular dystrophy is Becker Muscular Dystrophy (BMD).

In one embodiment, provided herein is a method for treating a muscle disease in a subject, comprising administering to the subject a composition that inhibits binding of a utrophin microRNA to the utrophin mRNA 3′-UTR, thereby treating the disease in the subject.

The term “treatment” or “treating,” as used herein, refers to any treatment of a disease in a mammal and includes: (1) preventing the disease from occurring in a subject which may be predisposed to the disease but does not yet experience or display symptoms of the disease; e.g., preventing the outbreak of clinical symptoms; (2) inhibiting the disease, e.g., arresting its development; or (3) relieving the disease, e.g., causing regression of symptoms of the disease.

An effective dosage to treat a disease means an amount which, when administered to a subject in need thereof, is sufficient to effect treatment, as defined above, of the disease. Treatment methods described herein can be used to treat a suitable mammal, preferably a human.

The term “subject,” as used herein, includes any human or non-human animal. The term “non-human animal” includes all vertebrates, e.g., mammals and non-mammals, such as non-human primates, sheep, dog, cow, chickens, amphibians, reptiles, etc.

In one embodiment, provided herein are methods of treating Duchene muscular dystrophy (DMD) in a subject, comprising: administering to the subject a composition that inhibits binding of a utrophin microRNA to the utrophin mRNA 3′-UTR, thereby treating DMD in the subject. In one embodiment, provided herein are methods of reducing signs and symptoms associated with Duchenne muscular dystrophy (DMD) in a subject, comprising: administering to the subject a composition that inhibits binding of a utrophin microRNA to the utrophin mRNA 3′-UTR, thereby reducing the symptoms associated with DMD in a subject.

In one embodiment, provided herein are methods of treating Becker muscular dystrophy (BMD) in a subject, comprising: administering to the subject a composition that inhibits binding of a utrophin microRNA to the utrophin mRNA 3′-UTR, thereby treating BMD in the subject. In one embodiment, provided herein are methods of reducing signs and symptoms associated with Becker muscular dystrophy (BMD) in a subject, comprising: administering to the subject a composition that inhibits binding of a utrophin microRNA to the utrophin mRNA 3′-UTR, thereby reducing symptoms associated with BMD in the subject.

In one embodiment, provided herein are composition comprising an effective amount of an agent that inhibits binding of a utrophin microRNA to the utrophin mRNA 3′-UTR. In one embodiment, the agent comprises a Let-7c antisense molecule (e.g., a sequence set forth in SEQ ID NOs: 1-5).

In one embodiment, a composition that inhibits utrophin microRNA binding induces utrophin protein production. In one embodiment, a utrophin microRNA molecule is a microRNA molecule that binds the 5′- or 3′-UTR of utrophin mRNA and inhibits utrophin protein production.

In one embodiment, administering a composition that inhibits utrophin microRNA binding comprises contacting a utrophin microRNA molecule with a utrophin microRNA antisense specific molecule. In one embodiment, a composition that inhibits utrophin microRNA binding comprises a utrophin microRNA antisense molecule. In one embodiment, a composition that inhibits utrophin microRNA binding comprises a utrophin microRNA antisense specific molecule. In one embodiment, a composition that inhibits a muscle cell specific microRNA molecule comprises a utrophin microRNA antisense specific molecule.

In one embodiment, a composition that inhibits utrophin microRNA binding is administered to a muscle cell in a subject. In one embodiment, a composition that inhibits utrophin microRNA binding is administered to a subject and is targeted to a muscle cell.

In one embodiment, methods described herein reduce signs and symptoms associated with Duchenne muscular dystrophy (DMD) or Becker muscular dystrophy (BMD). In one embodiment, methods described herein improve walking of a DMD or BMD patient. In one embodiment, methods described herein reduce or inhibit calves swelling with fibrous tissue. In one embodiment, methods described herein induce muscle growth. In one embodiment, methods described herein induce muscle regeneration. In one embodiment, methods described herein reduce or inhibit contractures. In one embodiment, methods described herein reduce or inhibit scoliosis. In one embodiment, methods described herein reduce or inhibit diaphragm weakening. In one embodiment, methods described herein reduce or inhibit a cardiac disease caused by or associated with lack of dystrophin.

The inhibitors and pharmaceutical compositions described herein can be administered to a subject by any suitable method known in the art, such as parenterally, paracancerally, transmucosally, transdermally, intramuscularly, intravenously, intra-dermally, subcutaneously, intra-peritonealy, intra-ventricularly, intra-cranially, intra-vaginally or intra-tumorally. In one embodiment, administration is systemic. In one embodiment, administration is intramuscular.

In one embodiment of methods and compositions described herein, the pharmaceutical compositions are administered orally, and are thus formulated in a form suitable for oral administration, i.e. as a solid or liquid preparation. Suitable solid oral formulations include tablets, capsules, pills, granules, pellets and the like. Suitable liquid oral formulations include solutions, suspensions, dispersions, emulsions, oils and the like. In one embodiment, the active ingredient is formulated in a capsule.

In one embodiment, the pharmaceutical compositions are administered by intravenous, intra-arterial, or intra-muscular injection of a liquid preparation. Suitable liquid formulations include solutions, suspensions, dispersions, emulsions, oils and the like. In one embodiment, pharmaceutical compositions are administered intravenously and are thus formulated in a form suitable for intravenous administration. In one embodiment, pharmaceutical compositions are administered intra-arterially and are thus formulated in a form suitable for intra-arterial administration. In one embodiment, pharmaceutical compositions are administered intra-muscularly and are thus formulated in a form suitable for intra-muscular administration.

In one embodiment, pharmaceutical compositions are administered topically to body surfaces and are thus formulated in a form suitable for topical administration. Topical formulations include, in one embodiment, gels, ointments, creams, lotions, drops and the like. In one embodiment, the pharmaceutical composition is administered as a suppository,

for example a rectal suppository or a urethral suppository. In one embodiment, the pharmaceutical composition is administered by subcutaneous implantation of a pellet. In one embodiment, the pellet provides for controlled release of active agent over a period of time.

In one embodiment, the active compound is delivered in a vesicle, e.g. a liposome. In some embodiments, carriers or diluents used in the compositions described herein

include, but are not limited to, a gum, a starch (e.g. corn starch, pregeletanized starch), a sugar (e.g. lactose, mannitol, sucrose, dextrose), a cellulosic material (e.g. microcrystalline cellulose), an acrylate (e.g. polymethylacrylate), calcium carbonate, magnesium oxide, talc, or mixtures thereof.

In other embodiments, pharmaceutically acceptable carriers for liquid formulations are aqueous or non-aqueous solutions, suspensions, emulsions or oils. Examples of non-aqueous solvents are propylene glycol, polyethylene glycol, and injectable organic esters such as ethyl oleate. Aqueous carriers include water, alcoholic/aqueous solutions, emulsions or suspensions, including saline and buffered media. Examples of oils are those of animal, vegetable, or synthetic origin, for example, peanut oil, soybean oil, olive oil, sunflower oil, fish-liver oil, another marine oil, or a lipid from milk or eggs.

In one embodiment, parenteral vehicles (for subcutaneous, intravenous, intra-arterial, or intramuscular injection) include sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Examples are sterile liquids such as water and oils, with or without the addition of a surfactant and other pharmaceutically acceptable adjuvants. In general, water, saline, aqueous dextrose and related sugar solutions, and glycols such as propylene glycols or polyethylene glycol are preferred liquid carriers, particularly for injectable solutions. Examples of oils are those of animal, vegetable, or synthetic origin, for example, peanut oil, soybean oil, olive oil, sunflower oil, fish-liver oil, another marine oil, or a lipid from milk or eggs.

In other embodiments, the compositions further comprise binders (e.g. acacia, cornstarch, gelatin, carbomer, ethyl cellulose, guar gum, hydroxypropyl cellulose, hydroxypropyl methyl cellulose, povidone), disintegrating agents (e.g. cornstarch, potato starch, alginic acid, silicon dioxide, croscarmelose sodium, crospovidone, guar gum, sodium starch glycolate), buffers (e.g., Tris-HCI., acetate, phosphate) of various pH and ionic strength, additives such as albumin or gelatin to prevent absorption to surfaces, detergents (e.g., Tween 20, Tween 80, Pluronic F68, bile acid salts), protease inhibitors, surfactants (e.g. sodium lauryl sulfate), permeation enhancers, solubilizing agents (e.g., glycerol, polyethylene glycerol), anti-oxidants (e.g., ascorbic acid, sodium metabisulfite, butylated hydroxyanisole), stabilizers (e.g. hydroxypropyl cellulose, hyroxypropylmethyl cellulose), viscosity increasing agents(e.g. carbomer, colloidal silicon dioxide, ethyl cellulose, guar gum), sweeteners (e.g. aspartame, citric acid), preservatives (e.g., Thimerosal, benzyl alcohol, parabens), lubricants (e.g. stearic acid, magnesium stearate, polyethylene glycol, sodium lauryl sulfate), flow-aids (e.g. colloidal silicon dioxide), plasticizers (e.g. diethyl phthalate, triethyl citrate), emulsifiers (e.g. carbomer, hydroxypropyl cellulose, sodium lauryl sulfate), polymer coatings (e.g., poloxamers or poloxamines), coating and film forming agents (e.g. ethyl cellulose, acrylates, polymethacrylates) and/or adjuvants.

In one embodiment, pharmaceutical compositions provided herein are controlled-release compositions, i.e. compositions in which the active compound is released over a period of time after administration. Controlled- or sustained-release compositions include formulation in lipophilic depots (e.g. fatty acids, waxes, oils). In one embodiment, the composition is an immediate-release composition, i.e. a composition in which of the active compound is released immediately after administration.

In one embodiment, the pharmaceutical composition is delivered in a controlled release system. For example, the agent may be administered using intravenous infusion, an implantable osmotic pump, a transdermal patch, liposomes, or other modes of administration. In one embodiment, a pump may be used (see Langer, infra; Sefton, CRC Crit. Ref. Biomed. Eng. 14: 201 (1987); Buchwald et al., Surgery 88: 507 (1980); Saudek et al., N. Engl. J. Med. 321: 574 (1989). In one embodiment, polymeric materials are used; e.g. in microspheres in or an implant. In yet one embodiment, a controlled release system is placed in proximity to the target cell, thus requiring only a fraction of the systemic dose (see, e.g., Goodson, in Medical Applications of Controlled Release, vol. 2, pp. 115-138 (1984); and Langer, Science 249: 1527-33 (1990)).

The compositions also include incorporation of the active material into or onto particulate preparations of polymeric compounds such as polylactic acid, polglycolic acid, hydrogels, etc, or onto liposomes, microemulsions, micelles, unilamellar or multilamellar vesicles, erythrocyte ghosts, or spheroplasts.) Such compositions will influence the physical state, solubility, stability, rate of in vivo release, and rate of in vivo clearance.

Also included herein are particulate compositions coated with polymers (e.g. poloxamers or poloxamines) and the compound coupled to antibodies directed against tissue-specific receptors, ligands or antigens or coupled to ligands of tissue-specific receptors.

Also comprehended herein are compounds modified by covalent attachment of water-soluble polymers such as polyethylene glycol, copolymers of polyethylene glycol and polypropylene glycol, carboxymethyl cellulose, dextran, polyvinyl alcohol, polyvinylpyrrolidone or polyproline. The modified compounds are known to exhibit substantially longer half-lives in blood following intravenous injection than do the corresponding unmodified compounds (Abuchowski et al., 1981; Newmark et al., 1982; and Katre et al., 1987). Such modifications may also increase the compound's solubility in aqueous solution, eliminate aggregation, enhance the physical and chemical stability of the compound, and greatly reduce the immunogenicity and reactivity of the compound. As a result, the desired in vivo biological activity may be achieved by the administration of such polymer-compound abducts less frequently or in lower doses than with the unmodified compound. In one embodiment, the methods described herein comprise administering an active

compound as the sole active ingredient. However, also encompassed within the scope of the methods of treating diseases and disorders described herein comprise administering the active compound in combination with one or more other therapeutic agents. In one embodiment, these agents are appropriate for the disease or disorder that is being treated, as is known in the art. In one embodiment, provided herein are methods of treating Duchenne Muscular Dystrophy (DMD) in a human subject, comprising: administering to the subject an effective amount of an oligonucleotide that (i) specifically hybridizes to a Let-7e microRNA binding sequence in a utrophin mRNA 3′-untranslated region (UTR) and (ii) inhibits binding of a Let-7c microRNA to the utrophin mRNA 3′-UTR. In one embodiment, the oligonucleotide comprises a nucleic acid sequence of one of SEQ ID NOs: 1-5. In one embodiment, the oligonucleotide has a sequence comprising SEQ ID NO: 5. In one embodiment, the oligonucleotide is a morpholino or phosphorodiamidate morpholino (PMO) oligonucleotide. In one embodiment, the oligonucleotide is between 24 and 29 nucleotides long. In one embodiment, the oligonucleotide is administered systemically. In one embodiment, the oligonucleotide is administered intramuscularly.

In one embodiment, the above method further comprises administering to the subject an effective amount of a second oligonucleotide that (i) specifically hybridizes to a second microRNA binding sequence in the utrophin mRNA 3′-UTR and (ii) inhibits binding of the second microRNA to the utrophin mRNA 3′-UTR. In one embodiment, the second microRNA can be miR-133b, miR-150, miR-196b, miR-206, or miR-296-5p.

In one embodiment, provided herein are pharmaceutical compositions comprising at least one pharmaceutically acceptable excipient and an oligonucleotide that specifically hybridizes to a Let-7c microRNA binding sequence in a utrophin mRNA 3′-untranslated region (UTR), wherein the oligonucleotide is present in an amount effective in a human subject to inhibit Let-7 microRNA binding to its utrophin mRNA 3′-UTR. In one embodiment, the oligonucleotide comprises a nucleic acid sequence of one of SEQ ID NOs: 1-5. In one embodiment, the oligonucleotide sequence comprises SEQ ID NO: 5. In one embodiment, the oligonucleotide is a morpholino or phosphorodiamidate morpholino (PMO) oligonucleotide. In one embodiment, the oligonucleotide is between 24 and 29 nucleotides long. In one embodiment, the composition is formulated for systemic administration to a subject. In one embodiment, the composition is formulated for intramuscular administration to a subject.

In one embodiment, the above composition further comprises at least one additional oligonucleotide that specifically hybridizes to at least one additional microRNA binding sequence in the utrophin mRNA 3′-UTR, wherein the additional microRNA can be miR-133b, miR-150, miR-196b, miR-206, or miR-296-5p, and wherein the additional oligonucleotide is present in an amount effective in a human subject to inhibit binding of the additional microRNA to its corresponding utrophin mRNA 3′-UTR binding sequence.

In one embodiment, provided herein are methods to enhance utrophin production in a subject, comprising: administering to the subject an oligonucleotide that (i) specifically hybridizes to a Let-7c microRNA binding sequence in a utrophin mRNA 3′-untranslated region (UTR) and (ii) inhibits binding of a Let-7c microRNA to the utrophin mRNA 3′-UTR. In one embodiment, the oligonucleotide comprises a nucleic acid sequence of one of SEQ ID NOs: 1-5. In one embodiment, the oligonucleotide is a morpholino or phosphorodiamidate morpholino (PMO) oligonucleotide.

Other features and advantages of this invention will become apparent from the detailed description, examples, and figures. It should be understood, however, that the detailed description and specific examples while indicating preferred embodiments are given by way of illustration only, since various changes and modifications within the spirit and scope of the invention will become apparent to those skilled in the art from this detailed description.

### EXAMPLE 1

### Results

### Let-7c PMO SBO Treatment Showed Increased Utrophin Expression in C2C12 Cells

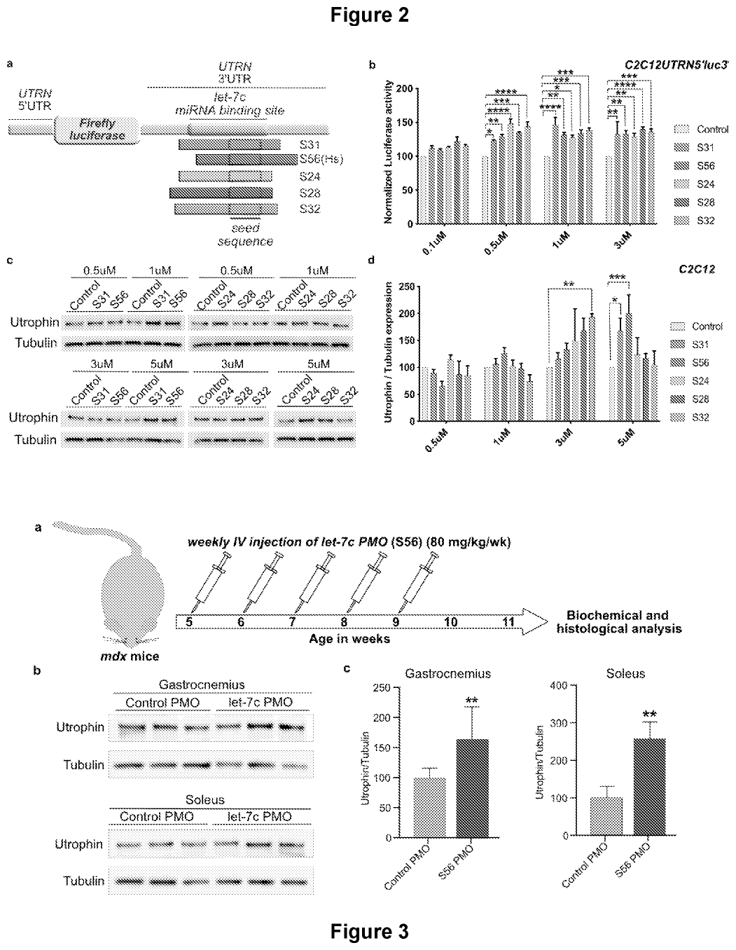

To determine the most efficient SBOs for achieving utrophin upregulation, five different Let-7c PMO SBOs (S24, S28, S31, S32 and S56) spanning different regions of the Let-7c target site in the UTRN 3′UTR were designed (FIG. 2A, Table 1). To evaluate the efficacy of these PMOs, the C2C12UTRN5′luc3′ reporter cell line containing the luciferase gene flanked by UTRN S′- and 3′-UTRs was used for reporter assays. Four different concentrations for each PMO were tested in the reporter cell line. S24 and S31 PMO-treated cells showed ˜1.5-fold higher luciferase activity compared to control PMO-treated cells at 0.5 μM and 1 μM concentrations, respectively (FIG. 2B). The higher activity of S24 and S31 PMO was noted in a narrow range of concentrations. However, S56 PMO, which was designed based on the human UTRN Let-7c sequence, showed ˜1.3-fold increase in luciferase activity from 0.5 μM to 3 μM concentration, implying effectiveness over a wider range of active concentrations. The S32 PMO also showed ˜1.4-fold increase in luciferase activity from 0.5 μM to 3 μM concentration (FIG. 2B, Table 3).

Whether the five PMOs could upregulate endogenous utrophin protein levels in C2C12 cells was further tested using western blotting as an independent, orthogonal assay. Consistent with the results obtained with the reporter cell line, S56 PMO had the highest level of utrophin expression (˜2-fold upregulation) compared to cells treated with control PMOs. Whereas the S32 PMO showed ˜1.9-fold higher utrophin expression at 3 μM but not at other concentrations (FIGS. 2C-2D, Table 4). Based on these findings, S56 PMO was chosen for an in vivo preclinical study in the mdx mouse DMD model. Mdx mice have a premature stop codon mutation in exon 23 of the DMD gene, and are widely used for preclinical animal studies for DMD.

### Systemic Administration of S56 PMO Upregulates Utrophin in Mdx Mice

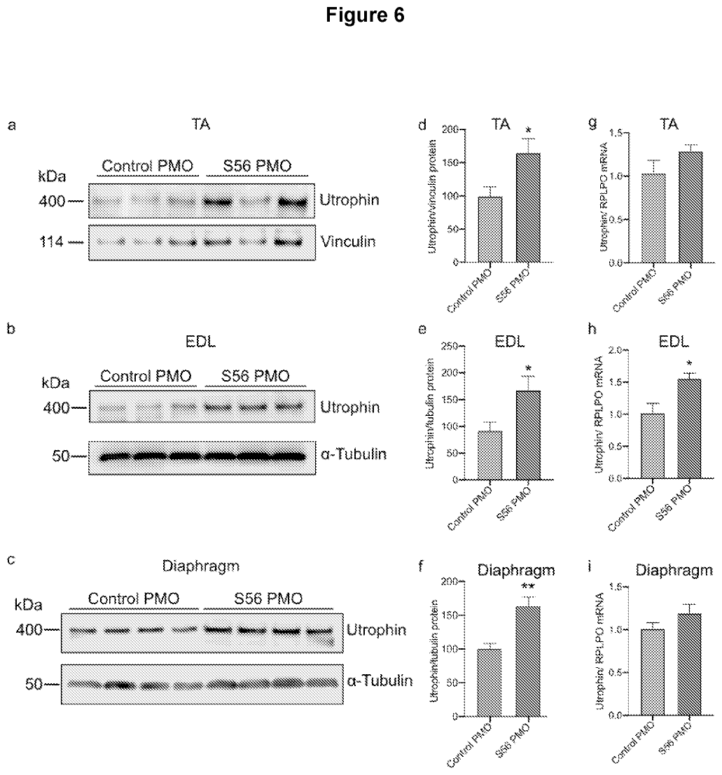

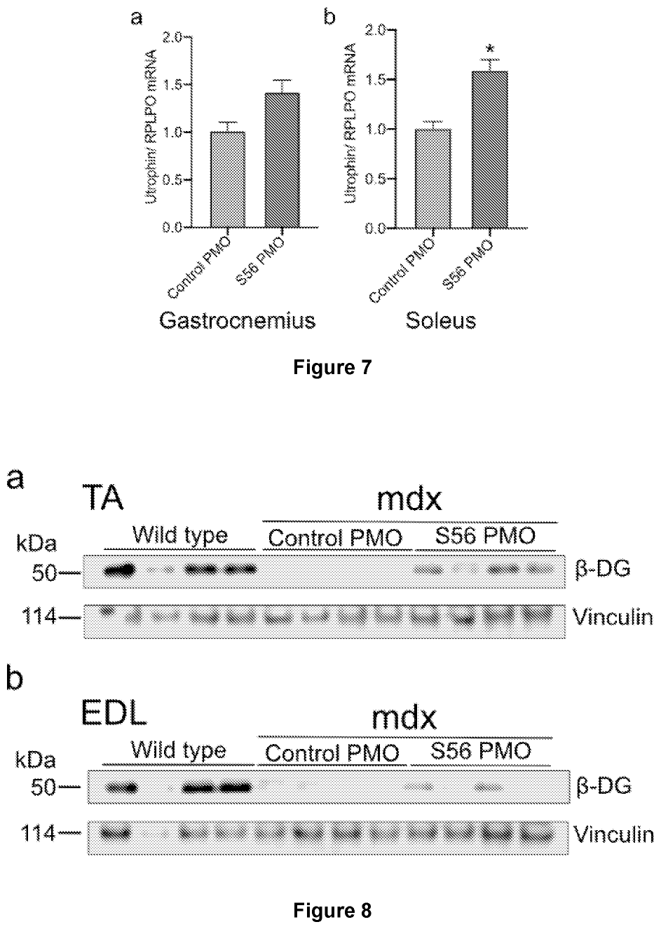

PK/pharmacodynamic (PD) studies of PMOs have shown highest bioavailability upon intravenous (iv) administration. Therefore, five weeks old mdx mice were treated with five weekly retro-orbital injections of S56 PMO or control PMO at a dosage of 80 mg/kg. Two weeks after treatment ended, the mice were sacrificed, and their skeletal muscles were examined (FIG. 3A). Western blots showed significant increases in utrophin protein expression in gastrocnemius (˜1.6-fold) and soleus (˜2.5-fold) muscles treated with S56 PMO compared to control PMO-treated mice (FIGS. 3B-3C). Surveyed muscles from S56 and control PMO-treated mice showed higher utrophin protein and mRNA expression levels, consistent with the intervention (FIGS. 6-7).

β-dystroglycan (β-DG) expression, which is a hallmark for restoration of dystrophin-glycoprotein complex (DGC), was tested. Western blots showed partial restoration of β-DG in S56 PMO-treated TA and EDL muscles (FIG. 8).

### S56 PMO Treatment Increased Sarcolemmal Utrophin Expression in Mdx Mice

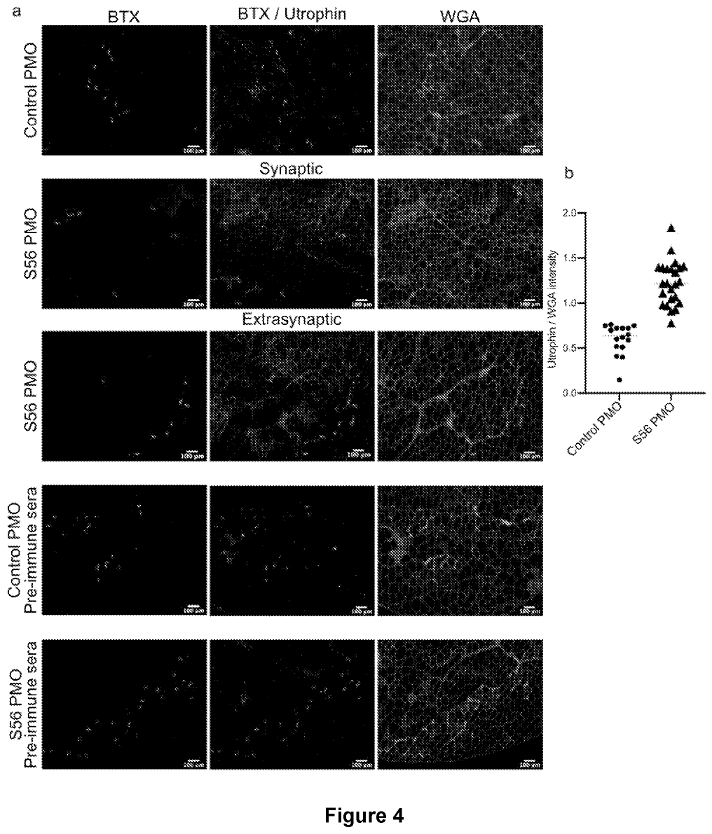

As western blots showed higher utrophin protein expression in S56 PMO-treated mdx mice, utrophin protein expression in the sarcolemma of control and S56 PMO-treated mdx mice was examined by immunohistochemistry. Control PMO-treated TA sections showed utrophin expression primarily restricted to neuromuscular junctions, as indicated by colocalization with the synaptic marker a-Bungarotoxin (BTX). On the other hand, muscles from mice treated with S56 PMO showed increased expression of utrophin across both synaptic and extrasynaptic regions (FIG. 4A). Quantification of utrophin level normalized to wheat germ agglutinin (WGA) levels in sarcolemma showed ˜2-fold higher expression of utrophin in TA muscles of S56 PMO-treated mdx mice (FIG. 4B).

### S56 PMO Treated Mdx Mice Showed Decreased Muscle Degeneration and Morphological Improvement

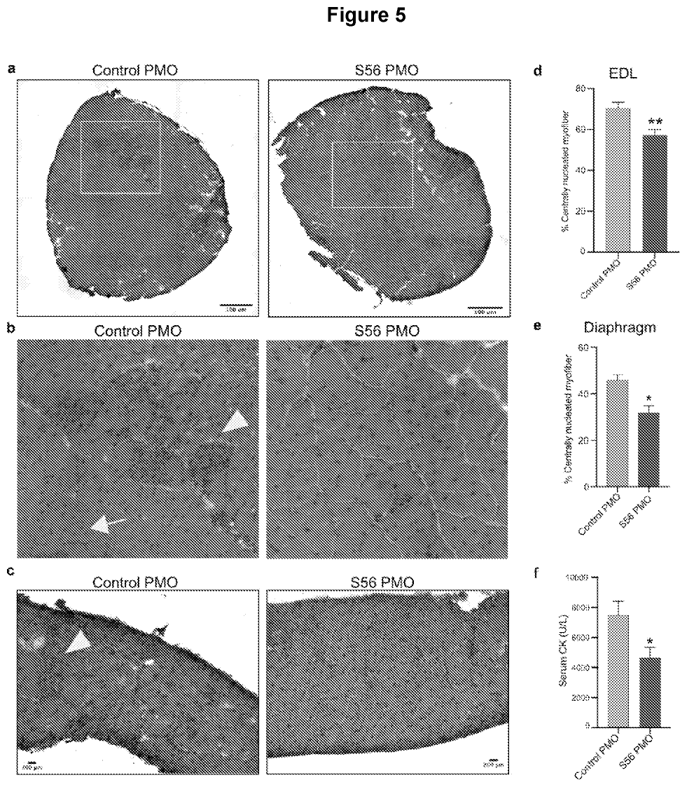

Whether the dystrophic histopathology was improved by S56 PMO treatment compared to control PMO treatment was further examined in mdx mice. H&E staining of EDL and diaphragm sections of control PMO-treated mdx mice showed regenerating centrally nucleated fibers (CNFs) and immune cell filtration as previously reported. However, Let-7c PMO treatment of age-matched mdx mice showed improvement in the pathophysiology, with reduced CNFs, necrosis and cellular infiltration observable in EDL and diaphragm (FIGS. 5A-5C). A higher percentage of CNFs is one of the well-recognized, characterized and objectively scored morphological characteristics of dystrophic muscles, attributed to continuous muscle regeneration. Two weeks after treatment ended, EDL and diaphragm muscles of mdx mice showed significantly lower percentage of CNFs in S56 PMO-treated mice (c.a. 13% reduction) compared to control PMO-treated mice, providing morphological evidence of improvement of the dystrophic phenotype (FIGS. 5D-5E). The morphological improvements noted in terms of reduction in the percentage of CNFs and muscle degeneration were not accompanied by changes in fiber size or contractile properties (Table 2).

### S56 PMO Treatment Decreases Serum CK Levels in Mdx Mice

The therapeutic effects of Let-7c PMO treatment was further studied in mdx mice by examining serum CK levels. Elevated CK level is one of the hallmarks of dystrophic pathophysiology in mice and humans. Two weeks after treatment ended, serum CK was significantly lower (c.a. 38% reduction) in S56 PMO-treated compared to control PMO-treated mdx mice (FIG. 5F). The decrease in serum CK provides biochemical evidence of utrophin mediated improvement in dystrophic pathology.

TABLE 1
PMO Sequences

| | | SEQ |
|---|---|---|
| PMO Oligo | Sequences 5′-3′ | ID NO: |
| S24 | CTGAGGTAGAAAGGTGATCATGGCTC | 1 |
| $28 | CTGAGGTAGAAAGGTGATCATGGCTCTCC | 2 |
| S31 | GTTCTGAGGTAGAAAGGTGATCATGGCTC | 3 |
| S32 | TCTGAGGTAGAAAGGTGATCATGGCTCT | 4 |
| S56 | AAGATGGATCTGAGGTAGAAAGGT | 5 |
| Control | GTGAGCACTTCTTTCCTTCTTTTTT | 6 |

TABLE 2
Comparison of Morphological And Physiological
Properties of EDL Muscle (N = 10)

| | 80 mg/kg/wk | | |
|---|---|---|---|
| | Control PMO | S56 PMO | P value |
| Weight (mg) | 13.3 ± 0.48 | 12.1 ± 0.37 | 0.06 |
| Cross sectional area (CSA) (mm²) | 2.36 ± 0.07 | 2.17 ± 0.06 | 0.03* |
| Absolute Tetanic force (mN) | 379.72 ± 17.7 | 362.15 ± 15.6 | 0.38 |
| Specific Tetanic force (N/cm²) | 16.10 ± 0.62 | 16.7 ± 0.57 | 0.86 |
| ECC force decrease (1-4) (%) | 77.26 ± 1.92 | 72.93 ± 1.86 | 0.06 |
| ECC force drop (4^{th}) (%) | 22.74 ± 1.92 | 27.07 ± 1.86 | 0.06 |
| Avg. of Minimal Feret's diameter (μm) | 31.58 ± 0.7 | 36.37 ± 2.29 | 0.4 |
| Variance coefficient of Min. Feret's diameter | 305.27 ± 14.5 | 321 ± 16.33 | 0.7 |

Results are presented as mean±SEM; S56 PMO-treated group was compared with control PMO-treated group and statistical significance were analyzed by Mann-Whitney test.

TABLE 3
Statistical Significance Data of Luciferase
Expression in C2C125′UTRN3′ Cells
Treated with Control and Let-7c PMO

| | | |
|---|---|---|
| Tukey's Multiple comparisons test | significance | Adjusted P value |

0.1 μM

| | | |
|---|---|---|
| Control vs. S31 | Ns | 0.6332 |
| Control vs. S56 | Ns | 0.8349 |
| Control vs. S24 | Ns | 0.5218 |
| Control vs. S28 | Ns | 0.0697 |
| Control vs. S32 | Ns | 0.3654 |

0.5 μM

| | | |
|---|---|---|
| Control vs. S31 | * | 0.0495 |
| Control vs. S56 | ** | 0.0068 |
| Control vs. S24 | **** | <0.0001 |
| Control vs. S28 | *** | 0.0006 |
| Control vs. S32 | **** | <0.0001 |

1 μM

| | | |
|---|---|---|
| Control vs. S31 | **** | <0.0001 |
| Control vs. S56 | ** | 0.0021 |
| Control vs. S24 | * | 0.0103 |
| Control vs. S28 | *** | 0.0009 |
| Control vs. S32 | *** | 0.0002 |

3 μM

| | | |
|---|---|---|
| Control vs. S31 | ** | 0.0018 |
| Control vs. S56 | ** | 0.0013 |
| Control vs. S24 | ** | 0.0072 |
| Control vs. S28 | **** | <0.0001 |
| Control vs. S32 | *** | 0.0004 |

TABLE 4
Statistical Significance Data of Utrophin Expression
in C2C12 Cells Treated with Control and Let-7c PMO

| | | |
|---|---|---|
| Tukey's Multiple comparisons test | significance | Adjusted P value |

0.5 μM

| | | |
|---|---|---|
| Control vs. S31 | ns | 0.9960 |
| Control vs. S56 | ns | 0.5696 |
| Control vs. S24 | ns | 0.9884 |
| Control vs. S28 | ns | 0.9928 |
| Control vs. S32 | ns | 0.9847 |

1 μM

| | | |
|---|---|---|
| Control vs. S31 | ns | 0.9997 |
| Control vs. S56 | ns | 0.8141 |
| Control vs. S24 | ns | >0.9999 |
| Control vs. S28 | ns | >0.9999 |
| Control vs. S32 | ns | 0.8614 |

3 μM

| | | |
|---|---|---|
| Control vs. S31 | ns | 0.9722 |
| Control vs. S56 | ns | 0.6111 |
| Control vs. S24 | ns | 0.4046 |
| Control vs. S28 | ns | 0.0947 |
| Control vs. S32 | ** | 0.0073 |

5 μM

| | | |
|---|---|---|
| Control vs. S31 | * | 0.0229 |
| Control vs. S56 | *** | 0.0002 |
| Control vs. S24 | ns | 0.9389 |
| Control vs. S28 | ns | 0.9853 |
| Control vs. S32 | ns | >0.9999 |

### Discussion

In this study, PMO-based Let-7c SBOs were developed and used to alleviate miRNA mediated repression of utrophin expression and achieved improved mdx pathophysiology in vivo. Utrophin mRNA translation is known to be inhibited by a set of six miRNAs. It had been shown that AAV mediated expression of a miR-206 sponge decoy in mdx mice, resulted in utrophin upregulation and improvement in the dystrophic phenotype, independently demonstrating the importance of miRNA mediated regulation of utrophin expression. The present disclosure shows higher utrophin expression and improvement of dystrophic phenotype by blocking the Let-7c target site in UTRN 3′UTR as compared to 2OMePS-based Let-7c SBO in vivo. Additionally, sPIF-mediated let-7 downregulation has also been shown to result in utrophin upregulation and improvement in the dystrophic phenotype, underscoring the significance of let-7 mediated regulation of utrophin expression. However, clinical application of 2OMePS-based Let-7c SBOs for utrophin upregulation or 2OMePS-based DMD exon skipping AONs is somewhat compromised by their suboptimal PKs and therapeutic efficacy. This disclosure tested the efficacy of five different Let-7c PMO-based SBOs for utrophin upregulation in vitro and studied the most efficient PMO (S56 PMO) in mdx mice to demonstrate improvement in dystrophic pathophysiology.

AAV mediated p-utrophin delivery and artificial transcriptional factor mediated

transcriptional upregulation of full length utrophin respectively, have been shown to improve dystrophic pathophysiology in the mdx model. Notably, p-utrophin expression does not induce significant immune responses compared to μ-dystrophin expression in deletional animal DMD models. In this study, weekly S56 PMO administration for 5 weeks resulted in ˜2.5-fold and ˜1.6-fold higher utrophin levels in soleus and gastrocnemius muscle respectively, biochemical and morphological improvement as demonstrated by a 38% reduction in serum CK levels and ˜13% reduction in percentage of CNFs compared to control PMO-treated mdx mice (FIGS. 3-5). Previous studies using two different doses (10 mg/kg and 100 mg/kg) of 2OMePS Let-7c SBO showed dose-dependent utrophin protein upregulation in skeletal and diaphragm muscle and improvement in mechanical properties only at the higher dose. However, even at the higher dose of 2OMePS Let-7c SBO, serum CK level showed no improvement. Here, with a relatively low dose of 80 mg/kg, S56 PMO treatment for five weeks in mdx mice showed increased utrophin expression, morphological improvement and a decrease in serum CK level. Higher doses of the lead PMO-based Let-7c SBO was not tested due to volume constraints of retrobulbar delivery, coupled with the high GC content of the target site, which limited S56 PMO SBO solubility. It is believed that designing and testing of additional SBOs and/or use of alternate chemistries (e.g., Pep-PMO, FANA) would help overcome this limitation.

It is believed that the functional improvement reported herein can be further improved with an earlier onset of treatment, a longer period of treatment and/or administration of higher

PMO dosages. Given that PMOs have shown sufficient safety and efficacy in vivo at higher doses (100-300 mg/kg/wk), the 80 mg/kg/wk used here should not be limiting. Additional sequences based on S56 could be tested to quantify dose-dependent improvements. Additionally, although one specific miRNA:utrophin interaction was targeted here, this approach can easily be extended to additional miRNAs that have been identified, either alone or in combination, to extend the potential benefits. Based on the findings reported here, it is proposed that PMO-based utrophin miRNA SBOs are a promising approach for achieving utrophin mediated therapeutics for DMD.

### Materials and Methods

### Cell Culture

C2C12 mouse myoblast cell lines (both from ATCC) and the C2C12UTRN5′luc3′ stable cell lines were maintained in standard growth condition in DMEM high glucose (Gibco, MD) supplemented with 10% fetal bovine serum (Sigma-Aldrich, MO) and 1% Pen/Strep (Gibco, MD).

### PMO-Based SBO Design

PMO backbone Let-7c SBOs were designed to specifically target and block the Let-7c miRNA target site in UTRN 3′UTR. Five PMOs were designed to target the same Let-7c site but with different flanking regions considering binding efficiency. A control PMO was designed with a scramble sequence. PMOs were synthesized by Gene Tools (OR) (Table 1).

### Luciferase Assay

The C2C12UTRN5′luc3′ cell line was seeded at 50,000 cells/well in 24 well plates and allowed to attach O/N. Next day, cells were treated for 24 hrs. with control or Let-7c PMOs of desired concentrations (0.1 μM, 0.5 μM, 1 μM, 3 μM) along with 6 μM transfection reagent endoporter (Gene Tools, LLC, OR). The next day, treated cells were lysed using the passive lysis buffer (Promega, WI) supplemented with complete protease inhibitor (Roche, Switzerland) and total protein contents were measured with the Pierce BCA protein assay kit (Thermo Fisher Scientific, MA). Luciferase assays were done using the luciferase assay system (Promega, WI) in a 96 well plate and quantified using a Cytation5 plate reader (BioTek, VT). Luciferase activity was normalized to the total protein loaded.

### Animal Studies

All animal procedures were reviewed and approved by the Institutional Animal Care and Use Committee at the University of Pennsylvania (UPenn). The C57BL/10ScSn-mdx/J mouse model of DMD (mdx) was utilized for all experiments. Breeding pairs were obtained from the Jackson Laboratory (Bar Harbor, ME). Mice were housed and bred at the UPenn animal facility, provided food, water ad libitum, and maintained under 12 hrs. light/dark cycles.

### Preclinical Studies in Mdx Mice with PMOs

PMOs were solubilized in saline and warmed at 50° C. for 15 mins and cooled down to room temperature before injections. Five-week-old male mdx mice were anesthetized with 4% isoflurane and control or S56 PMO was administered systemically via the retro-orbital sinus as a single 80 mg/kg dose (<250 μL volume) using a 28-gauge/0.5 mL insulin syringe, weekly for 5 weeks. Two weeks after the last injection, mice were anesthetized by 4% isoflurane and sacrificed. Ex vivo contractility analyses were done as previously described. Following the procedure, muscles were flash-frozen in liquid nitrogen-cooled isopentane and stored at −80° C.

### Tissue Harvesting and Cryosectioning

Muscle samples were surgically removed after euthanasia, embedded in OCT, flash frozen in liquid nitrogen-chilled isopentane and stored at −80° C. For immunohistochemistry and H&E staining, tissues were sectioned at 5 um thickness on a Leica CM1950 cryostat and stored at −80° C.

### Immunohistochemistry

Cryosections were blocked for 1 hour with 4% BSA in PBS, followed by staining with custom made rabbit anti-utrophin A antibody raised with the N-terminal utrophin A peptide (CMAKYGDLEARPDDGQNE, SEQ ID NO: 12) as described before (1:500, ProSci Inc, CA) overnight at 4° C. in a humidified chamber. Sections were then washed and incubated with a goat anti-rabbit Alexa Fluor 488 conjugated secondary antibody (Life Technologies, MA) at a dilution of 1:400 for 1 hour at room temperature. α-Bungarotoxin and Wheat germ agglutinin (WGA) conjugated to Alexa Fluor 594 or 647 (Life Technologies, MA) were used at a dilution of 1:400 for 30 mins at room temperature. Sections were mounted with Prolong Gold Antifade Mounting Media with DAPI (Thermo Fisher Scientific, MA) for nuclear staining.

### Western Blots

Frozen tissue samples were cut with pre-cooled scissors and minced in tubes pre-cooled in dry ice. Samples were then suspended in muscle denaturing buffer (50 mM Tris-HCl pH 7.5, 150 mM NaCl, 1% Triton X100, 1 mM EDTA, 10 mM MgCl₂, 1% SDS, 0.5 mM DTT, 10% glycerol) with complete protease inhibitor cocktail (Roche, Basel, Switzerland). Equilibrated tissue samples were homogenized using a TissueLyser II (Qiagen, Hilden, Germany) with 5 mm stainless steel beads (Thermo Fisher Scientific, MA) at a 20 Hz frequency for 2 mins. C2C12 cells were lysed with RIPA buffer (Thermo Fisher Scientific, MA) with protease inhibitor cocktail. Total protein was measured by Pierce BCA Protein assay kit (Thermo Fisher Scientific, MA). Ten μg of total protein was resolved in 3-8% Tris-Acetate protein gel (NuPAGE, Thermo Fisher Scientific, MA) and transferred to a nitrocellulose membrane using Trans-Blot Turbo Transfer System (Bio-Rad, CA). For western blotting, membranes were first blocked with 5% non-fat dry milk in TBS with 1% Tween 20 for 1 hr. at room temperature. After blocking, blots were incubated with the following primary antibodies; mouse monoclonal anti-utrophin (1:100, Mancho3(8A4), developed by Prof. Glenn E. Morris; DSHB, Iowa) and mouse anti-α-Tubulin (1:5000, T6199, Sigma-Aldrich, MO) overnight at 4°C. Next day, blots were washed; incubated with mouse IgGκ binding protein (m-IgGκ BP) conjugated to horseradish peroxidase (HRP) (1:2500, sc-516102, Santa Cruz Biotechnology, TX); washed and developed using Immobilon Western Chemiluminescent HRP Substrate (MilliporeSigma, MA) and imaged in LI-COR C-Digit Blot Scanner (LI-COR Biosciences, NE).

### Serum CK Assay

Blood samples were collected via cardiac puncture under deep terminal anesthesia, centrifuged at 2000 g for 5 mins and serum stored at −80° C. until analysis. CK levels were determined by the Clinical Pathology Laboratory at the Matthew J. Ryan Veterinary Hospital of the University of Pennsylvania.

### Total RNA Isolation. Reverse Transcription And Quantitative Real Time PCR (qPCR) From Mdx Mice Tissue Samples

Frozen tissue samples were cut with pre-cooled scissors and minced in tubes pre-cooled in dry ice. Samples were then suspended with 500 μL of TRIzol reagent (Invitrogen) and homogenized using a TissueLyser II (Qiagen, Hilden, Germany) with 5 mm stainless steel beads (Thermo Fisher Scientific, MA) at a frequency of 20 Hz for 2 mins. Total RNA was harvested following manufacturer's protocol. 1 μg of RNA from each sample was reverse transcribed with SuperScript™ III Reverse Transcriptase (Thermo Fisher Scientific, MA) using random hexamer following manufacturer's protocol. Quantitative PCR was performed in QuantStudio3 Real-Time PCR System (Applied Biosystems) using Power SYBR Green Master Mix (Applied Biosystem) as described before.

### Statistical Analysis

Data were analyzed using the GraphPad Prism v8 statistical software package. Values are presented as mean±standard error of the mean (SEM). Statistical analysis was performed using Mann-Whitney test or Two-Way ANOVA multiple comparisons with statistical significance level set at α≤0.05.

What is claimed is:

1. A method of treating Duchenne Muscular Dystrophy (DMD) in a human subject, the method comprising administering to said subject an effective amount of an oligonucleotide that (i) specifically hybridizes to a Let-7e microRNA binding sequence in a utrophin mRNA 3′-untranslated region (UTR) and (ii) inhibits binding of a Let-7c microRNA to the utrophin mRNA 3′-UTR.

2. The method of claim 1, wherein the oligonucleotide comprises a nucleic acid sequence selected from the group consisting of SEQ ID NOs: 1-5.

3. The method of claim 1, wherein the oligonucleotide has a sequence comprising SEQ ID NO: 5.

4. The method of any one of claims 1-3, wherein the oligonucleotide is a morpholino or phosphorodiamidate morpholino (PMO) oligonucleotide.

5. The method of any one of claims 1-4, wherein the oligonucleotide is between 24 and 29 nucleotides long.

6. The method any one of claims 1-5, wherein the oligonucleotide is administered systemically.

7. The method any one of claims 1-5, wherein the oligonucleotide is administered intramuscularly.

8. The method of claim 1, further comprising the step of administering to said subject an effective amount of a second oligonucleotide that (i) specifically hybridizes to a second microRNA binding sequence in the utrophin mRNA 3′-UTR and (ii) inhibits binding of the second microRNA to the utrophin mRNA 3′-UTR, wherein said second microRNA is selected from the group consisting of miR-133b, miR-150, miR-196b, miR-206, and miR-296-5p.

9. A pharmaceutical composition comprising at least one pharmaceutically acceptable excipient and an oligonucleotide that specifically hybridizes to a Let-7c microRNA binding sequence in a utrophin mRNA 3′-untranslated region (UTR), wherein the oligonucleotide is present in an amount effective in a human subject to inhibit binding of Let-7 microRNA to its utrophin mRNA 3′-UTR.

10. The composition of claim 9, wherein the oligonucleotide comprises a nucleic acid sequence selected from the group consisting of SEQ ID NOs: 1-5.

11. The composition of claim 9, wherein the oligonucleotide has a sequence comprising SEQ ID NO: 5.

12. The composition of any one of claims 9-11, wherein the oligonucleotide is a morpholino or phosphorodiamidate morpholino (PMO) oligonucleotide.

13. The composition of any one of claims 9-12, wherein the oligonucleotide is between 24 and 29 nucleotides long.

14. The composition of any one of claims 9-13, wherein the composition is formulated for systemic administration to a subject.

15. The composition of any one of claims 9-13, wherein the composition is formulated for intramuscular administration to a subject.

16. The composition of claim 9, further comprising at least one additional oligonucleotide that specifically hybridizes to at least one additional microRNA binding sequence in the utrophin mRNA 3′-UTR, wherein the additional microRNA is selected from the group consisting of miR-133b, miR-150, miR-196b, miR-206, and miR-296-Sp, and wherein the additional oligonucleotide is present in an amount effective in a human subject to inhibit binding of the additional microRNA to its corresponding utrophin mRNA 3′-UTR binding sequence.

17. A method for enhancing utrophin production in a subject, the method comprising administering to said subject an oligonucleotide that (i) specifically hybridizes to a Let-7c microRNA binding sequence in a utrophin mRNA 3′-untranslated region (UTR) and (ii) inhibits binding of a Let-7c microRNA to the utrophin mRNA 3′-UTR.

18. The method of claim 17, wherein the oligonucleotide comprises a nucleic acid sequence selected from the group consisting of SEQ ID NOs: 1-5.

19. The method of claim 17 or claim 18, wherein the oligonucleotide is a morpholino or phosphorodiamidate morpholino (PMO) oligonucleotide.
